(19) United States
(12) Patent Application Publication
(10) Pub. No.: US 2008/0038384 A1
(43) Pub. Date: Feb. 14, 2008

(54) Formulation to aid in management of Irritable Bowel Syndrome with urgency symptoms.

(76) Inventor: Howard Sachs, South Orange, NJ (US)

Correspondence Address: Howard Sachs;DWD Consumer Products, Inc., #275, 623 Eagle Rock Avenue, West Orange, NJ, 07052, US

(21) Appl. No.: 11/834,659

(22) Filed: Aug. 6, 2007

Related U.S. Application Data
(60) Provisional application No. 60/837,601, filed on Aug. 11, 2006.

(51) Int. Cl. A61K 36/534; A61K 36/268; A61P 1/00

(52) U.S. Cl. 424/747; 424/756

(57) ABSTRACT

A formulation designed to help sufferers of Irritable Bowel Syndrome who are prone to urgency symptoms. The formulation contains herbs, calcium carbonate, and food ingredients and the combination aids in management of the condition.

### CROSS-REFERENCE TO RELATED APPLICATIONS

This application claims the benefit of provisional patent application No. 60/837,601, filed Aug. 11, 2006 by the present inventor.

### FEDERALLY SPONSORED RESEARCH

Not applicable

### SEQUENCE LISTING OR PROGRAM

Not applicable

### FIELD OF THE INVENTION

The present invention relates generally to a formulation of herbs, minerals, and food. More particularly, the invention is directed to a mixture of herbs, minerals, and foods that can ease or pre-empt some symptoms of Irritable Bowel Syndrome (“IBS”).

### BACKGROUND OF THE INVENTION

IBS is a chronic gastrointestinal condition that has no cure. Approximately 1 in 5 adults suffer from IBS (>55 MM people in the U.S. alone) and it is responsible for more than 10% of all doctor visits and half of all GI complaints. IBS is the most common chronic health disorder in America, Canada, the UK, Australia, and New Zealand.

IBS is characterized by continuous or recurring lower abdominal pain or cramping. The pain can range from mild to excruciating and can prevent sufferers from going to work or caring for their families. This pain occurs in association with altered bowel motility (diarrhea, constipation, or both).

IBS-D—the colon does not absorb enough water, diarrhea results

IBS-C—the colon absorbs too much water, constipation results

IBS-A—alternating diarrhea and constipation (also referred to as IBS C/D)

The focus of this application is the sufferers of IBS-D and IBS-A. These people have attacks that can be very painful, that are unpredictable, and may involve severe and recurrent diarrhea. This plays havoc with sufferers' ability to work and care for others. It can also impact their quality of life as the fear of having to find a restroom immediately, and stay there through a long, painful attack, can cause them to avoid travel, restaurants, or social occasions. There is no portable product that these IBS sufferers can carry with them to use when not feeling right. This invention seeks to address that need, among others.

### PRIOR ART

Prior inventions (see e.g. U.S. Pat. No. 4,689,667) have attempted to ease IBS symptoms through products that are enteric coated so that they do not begin dissolving in the stomach. They deliver their product to the intestines and relieve pain by relaxing the muscle of the intestine. In addition some prior inventions sought to treat IBS by inhibiting gastric secretions (see, e.g. U.S. Pat. No. 6,156,771).

### SUMMARY OF THE INVENTION

This formulation combines peppermint oil, ginger, fennel, calcium carbonate, and dextrose to deliver a surprising and synergistic combination of beneficial results to an IBS sufferer who is prone to urgency-related symptoms such as diarrhea.

### DRAWINGS

Not necessary for this invention.

### DETAILED DESCRIPTION OF THE PREFERRED EMBODIMENT

The present invention is comprised of peppermint oil, ginger, fennel, calcium carbonate, and dextrose. This product is useful as a food supplement, particularly for those sufferers of IBS who experience urgency symptoms.

The preferred embodiment is designed to begin digesting in the stomach. This product is slightly counterintuitive and contrarian to prior art as it attacks IBS symptoms in the opposite manner of prior inventions. In direct opposition to prior art, this formulation (current embodiment is a chewable tablet) is designed to begin dissolving in the stomach to, among other things, ease the gastrocolic reflex (the stomach's message to the intestines and which may be quite unpredictable and severe in IBS sufferers). In addition, in opposition to prior art, this invention encourages rather than inhibits gastric secretion. Prior inventions seek to treat IBS symptoms occurring in the intestines, this invention seeks to reduce or stop those symptoms before they start. The combination of ingredients has an unexpected and synergistic combination of benefits for an EBS sufferer.

Peppermint has anti-spasmodic qualities, relaxing muscles of the digestive system. It has anesthetic and anti-inflammatory effects, helping to reduce pain and soothe the nerves of the digestive system. It stimulates bile and gastric secretions to help digestion and has carminative (anti-gas) properties. It relieves diarrhea. Peppermint oil may be present in the inventive formulation at a concentration of 0.5 to 5 weight percent.

Ginger contains powerful digestive enzymes and stimulates gastric secretions to help digestion. It is antispasmodic and anti-inflammatory. Ginger may be present in the inventive formulation at a concentration of 0.2 to 5 weight percent.

Fennel seed prevents gas, inhibits spasms in the smooth muscles of the digestive system, and reduces pain. It fights bloating. It helps regulate contractions of the small intestine. Fennel seed may be present in the inventive formulation at a concentration of 0.2 to 4 weight percent.

Calcium carbonate has a constipating effect, and antacid effect, and is beneficial for the colon. It may also counteract heartburn (which can be caused by peppermint oil). Calcium carbonate may be present in the inventive formulation at a concentration of 5 to 25 weight percent.

Dextrose is a sugar. Research indicates that up to 25% of IBS sufferers have problems tolerating fructose. This has become a bigger issue over the past 17 years, as almost all processed food has transitioned to replacing table sugar with high fructose corn syrup. There are indications that ingesting dextrose may help these sufferers to digest fructose. Dextrose may be present in the inventive formulation at a concentration of 0.2 to 25 weight percent.

The formulation may also contain conventional food supplements that bind, preserve, coat, improve texture, or change taste/flavor.

The ingredients may be dried, ground, and mixed together by conventional techniques. The mixture may then be pressed into a pressed tablet. The current tablet is 965 mg and may be taken as needed. While the current and preferred embodiment is a chewable tablet, it may be developed in other forms, including but not limited to liquid, chew, capsule, strip, or gum without departing from its spirit and scope.

1. A formulation for dietary management of Irritable Bowel Syndrome, which is comprised of peppermint oil and ginger.

2. The formulation according to claim 1, further including fennel.

3. The formulation according to claim 1, further including calcium carbonate.

4. The formulation according to claim 1, further including dextrose.

5. The formulation according to claim 1, further including fennel, calcium carbonate and dextrose.

6. The formulation according to claim 1, where the concentration of peppermint oil ranges from about 0.5 weight percent to 5 weight percent of the formulation.

7. The formulation according to claim 1, where the concentration of ginger ranges from about 0.2 weight percent to 5 weight percent of the formulation.

8. The formulation according to claim 1, where the concentration of fennel ranges from about 0.2 weight percent to 4 weight percent of the formulation.

9. The formulation according to claim 1, where the concentration of calcium carbonate ranges from about 5 weight percent to 25 weight percent of the formulation.

10. The formulation according to claim 1, where the concentration of dextrose ranges from about 0.2 weight percent to 25 weight percent of the formulation.
